(19) United States
(12) Patent Application Publication
(10) Pub. No.: US 2016/0010125 A1
(43) Pub. Date: Jan. 14, 2016

(54) METHODS AND SYSTEMS FOR THE SIMULTANEOUS PRODUCTION OF LIPIDS AND AROMATICS FROM CELLULOSE FEEDSTOCKS

(71) Applicant: Menon Renewable Products, Inc., San Diego, CA (US)

(72) Inventors: Suresh M. Menon, San Diego, CA (US); David E. Newman, Poway, CA (US); Jagadish Chandra Sircar, San Diego, CA (US); Kashinatham Alisala, San Diego, CA (US); Kay A. Yang, San Diego, CA (US); Samantha Orchard, San Diego, CA (US); Sara Guidi, San Diego, CA (US)

(21) Appl. No.: 14/660,669

(22) Filed: Mar. 17, 2015

Related U.S. Application Data
(63) Continuation of application No. 12/573,732, filed on Oct. 5, 2009, now abandoned.
(60) Provisional application No. 61/213,906, filed on Jul. 28, 2009.
(60) Provisional application No. 61/202,288, filed on Feb. 13, 2009.
(60) Provisional application No. 61/136,860, filed on Oct. 9, 2008.

(51) Int. Cl. C12P 7/64; C11B 3/00

(52) U.S. Cl. CPC C12P 7/6409; C11B 3/003; C12P 7/6427

(57) ABSTRACT

A system and method are provided which utilize microbes to convert biomass feedstock into a fuel. In one aspect, a method of producing lipids includes receiving a feedstock including biomass, exposing the feedstock to microbes which are capable of converting the feedstock into lipids, and extracting produced lipids.

### RELATED APPLICATIONS

This application claims the benefit of U.S. provisional application Ser. No. 61/213,906, filed Jul. 28, 2009, entitled “Microbial Processing of Cellulosic Feedstocks for Fuel,” Ser. No. 61/202,288, filed Feb. 13, 2009, entitled “Biofuels from Cellulose,” and Ser. No. 61/136,860, filed Oct. 9, 2008, entitled “Biofuels from Glycerol,” which are hereby incorporated by reference.

### BACKGROUND

1. Field of the Invention

The present application generally relates to the use of microbial and chemical systems to convert cellulosic and other biological waste materials to commodity chemicals, such as biofuels/biopetrols.

2. Related Art

Petroleum is facing declining global reserves and contributes to more than 30% of greenhouse gas emissions driving global warming. Annually 800 billion barrels of transportation fuel are consumed globally. Diesel and jet fuels account for greater than 50% of global transportation fuels.

Significant legislation has been passed, requiring fuel producers to cap or reduce the carbon emissions from the production and use of transportation fuels. Fuel producers are seeking substantially similar, low net carbon fuels that can be blended and distributed through existing infrastructure (e.g., refineries, pipelines, tankers).

Due to increasing petroleum costs and reliance on petrochemical feedstocks, the chemicals industry is also looking for ways to improve margin and price stability, while reducing its environmental footprint. The chemicals industry is striving to develop greener products that are more energy, water, and CO₂ efficient than current products. Fuels produced from biological sources represent one aspect of process.

### SUMMARY

A system and method are provided which utilize microbes to convert biomass feedstock into fuel.

In one aspect, a method of producing lipids includes receiving a feedstock including biological waste material, exposing the feedstock to microbes which are capable of converting the feedstock into lipids, and extracting produced lipids.

In one aspect of the invention, a method of producing fuel includes receiving a feedstock including cellulose, converting at least a portion of the feedstock into lipids using microbes, extracting the produced lipids from the microbes, and converting the produced lipids into liquid fuel.

In another aspect of the invention, a system for producing lipids includes a fermentor and a controller in communication with the fermentor. The controller provides operating instructions to the fermentor and the fermentor yields the lipids.

Other features and advantages of the invention will be apparent from the following detailed description, the claims and the appended drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a flow chart of a cellulosic feedstock pretreatment process according to an embodiment of the invention.

FIG. 2 is a flow chart of an inoculation and fermentation process according to an embodiment of the invention.

FIG. 3 is a flow chart of a microbe collection process according to an embodiment of the invention.

FIG. 4 is a flow chart of an inoculation and fermentation process according to an embodiment of the invention.

FIG. 5 is a flow chart of a separation process according to an embodiment of the invention.

FIG. 6 is a block diagram of process equipment used in accordance with FIGS. 1-5.

### DETAILED DESCRIPTION

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

The described embodiments relate to systems and methods for production of liquid fuel from low-value starting materials of biological origin. In some embodiments, the systems and methods relate specifically to the production of diesel, gasoline and/or aviation fuel from cellulosic feedstocks. In some embodiments, the method includes a multi-step process that inputs raw feedstock and outputs triacylglyceride (“TAG”) or other lipids, and aromatic compounds.

Present methods of converting cellulosic materials utilize feedstock specifically cultivated for producing biofuels. In addition to these “cultivated cellulosic feedstocks”, cellulosic feedstock may be obtained from cellulosic waste materials such as sawdust, wood chips, cellulose, algae, other biological materials, municipal solid waste (e.g., paper, cardboard, food waste, garden waste, etc.), and the like.

A process in accordance with an embodiment of the present invention includes converting cellulosic waste materials into liquid fuel. In one aspect, cellulosic material such as agricultural waste is converted into lipids such as TAG, using specially selected or developed microbes. These microbes convert free sugars, cellulose and hemicellulose, major components of plant matter, into TAG.

TAG includes three fatty acids linked to a glycerol backbone. When dissociated from the glycerol and hydrotreated, the fatty acids are converted to hydrocarbons, which form the major components of diesel, gasoline and jet fuel. In some embodiments, TAG itself may serve as a component of fuel. In other embodiments, the fatty acids are converted to fuel such as bio-diesel. A benefit associated with the present process is that no net carbon is added to the atmosphere when the fuel is burned because the feedstock was originally produced by photosynthesis, sequestering carbon dioxide from the atmosphere.

Gasoline and jet fuel specifications require, in addition to alkanes, a certain proportion of aromatic compounds. TAG cannot be readily converted to aromatic compounds. However, plant matter also contains lignin, a polymeric agglomeration of aromatic compounds that can be broken down into the aromatics required for fuel. Specialized microbes attack lignin and convert it into smaller, individual aromatic compounds. Thus, microbial conversion processes can suffice to convert agricultural and municipal waste originating from plant matter (paper, pulp, food waste, yard waste, etc.) into all the components of fuel.

In accordance with an embodiment of the present invention, a suitable biological feedstock includes high-molecular-weight, high-energy-content molecules such as sawdust, wood chips, cellulose, algae, other biological materials, or other solid materials to be converted into fuel. The resulting fuel may be in fluid form, meaning that gaseous and liquid components may contribute to the make up of the fuel. For example, in one embodiment, the resulting fuel may include methane (gas) and octane (liquid), as well as a variety of other components. The feedstock material may be a low-value or waste material.

In certain embodiments of the present invention, a cellulosic feedstock includes at least 10% cellulosic waste materials. In some embodiments, the cellulosic biomass feedstock includes greater than 50% cellulosic waste materials. In still other embodiments, the cellulosic biomass feedstock includes up to 100% cellulosic waste materials.

In one aspect, the feedstock may be a biological product of plant origin, thus resulting in no net increase in atmospheric carbon dioxide when the resultant fuel product is combusted.

In some embodiments, two or more feedstocks may be used. For example, a secondary feedstock may include any material by-product of a cellulose conversion process, which material is capable of being converted into fuel by microbial action. The secondary feedstock may include glycerol molecules or fragments thereof, or glycerol with additional carbon atoms or short paraffinic chains attached. Such compounds can be produced, for example, when alkanes are cleaved from TAG.

For simplicity of explanation, a process in accordance with the present invention may be divided into three main steps: (1) feedstock pretreatment, (2) inoculation and fermentation/digestion, and (3) harvesting and extraction of the lipids and/or aromatic products.

### (1) Feedstock Pretreatment

In an embodiment, raw feedstock is pretreated to make its carbon content accessible to microbial digestion and to kill any naturally present microbes that might compete with the preferred species introduced for the purpose of lipid and/or aromatic compound production. Pretreatment can include three steps: (1) mechanical pretreatment, (2) thermal-chemical pretreatment and sterilization or ultraviolet (“UV”) irradiation or pasteurization, and (3) filtration/separation. In the mechanical pretreatment step, raw feedstock may be conveyed to a chopper, shredder, grinder or other mechanical processor to increase the ratio of surface area to volume.

The thermal-chemical pretreatment step can treat the mechanically processed material with a combination of water, heat and pressure. Optionally, acidic or basic additives or enzymes may also be added prior to heat-pressure treatment. This treatment further opens up the solid component (e.g., increases the ratio of surface area to volume) for microbial access and dissolves sugars and other compounds into a liquid phase to make it more amenable to microbial digestion. Examples of such treatment include the class of processes known variously as hydrolysis or saccharification, but lower-energy processing, such as simple boiling or cooking in water, may also be utilized.

In one embodiment, non-carbon microbial nutrients are added prior to the thermal-chemical pretreatment step. Non-carbon microbial nutrients include, for example, sources of nitrogen, phosporous, sulfur, metals, etc. After adding the non-carbon microbial nutrients, the entirety may then be sterilized.

The filtration/separation step preferably separates the solid matter (e.g., where the lignin is concentrated) from the liquid (e.g., which contains most of the sugars and polysaccharides from the cellulose and hemicellulose in the feedstock).

In some embodiments, the feedstock is fortified (e.g., via the addition of glycerol.) For example, glycerol used in the feedstock fortification may be obtained as a byproduct of some TAG conversion processes. In particular, glycerol is released by the conversion of TAG to produce bio-diesel fuel (e.g. via transesterification). The released glycerol may then be metabolized to contribute to TAG formation. A benefit of adding glycerol to the feedstock is that it can speed the growth of certain microbial species during fermentation, discussed below. It is understood that glycerol obtained from transesterification is not high-purity, but rather includes a variety of constituents.

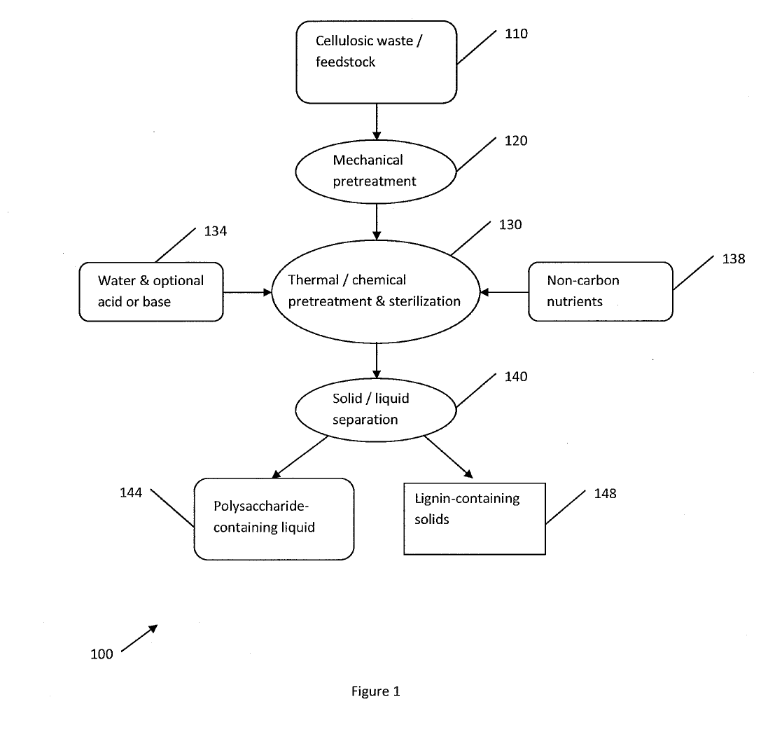

Referring now to FIG. 1, a flow chart of a cellulosic feedstock pretreatment process 100 in accordance with an embodiment of the invention is shown. The pretreatment process 100 includes a receiving stage 110 for receiving the cellulosic feedstock and a mechanical pretreatment stage 120 for transforming the feedstock into small particles.

The pretreatment process 100 also includes a thermo-chemical pretreatment stage 130 to open up the cellulosic structure, rendering the cellulosic structure more accessible to the microbes and to bring some of the sugars and polysaccharides into solution. In some embodiments, water and, optionally, acidic or basic additives 134 are added to the feedstock during this thermo-chemical pretreatment stage 130. In some embodiments, non-carbon nutrients 138 used for the microbial metabolization are also added during this thermo-chemical pretreatment stage 130. It should be appreciated that the thermo-chemical treatment step 130 also serves to sterilize the cellulosic material and surrounding liquid to inhibit potentially competing microorganisms.

The pretreatment process 100 also includes a solid-liquid separation stage 140 which may use mechanical means such as filters and/or centrifuges to separate the bulk of the solid feedstock from the liquid portion. As described above, the liquid portion 144 includes mostly sugars and polysaccharides, while the solid portion 148 includes lignin as well as undissolved cellulose and hemicellulose.

### (2) Inoculation and Fermentation

In the inoculation and fermentation stage, the solid and liquid portions of the treated feedstock are preferably placed in separate digesters. The digesters are vessels containing the feedstock material and microbes which break down the feedstock into lipids or aromatics, respectively, a solvent (e.g., water), and non-carbon nutrients (e.g., nitrates, phosphates, trace metals, and the like).

The microbes may be species of any of two classes: one class which converts cellulose, hemicellulose or glycerol into lipids, and a second class which breaks lignin down into aromatic compounds. Microbes including bacterial and/or fungal species which convert cellulose, hemicellulose or glycerol into lipids include, for example, Trichoderma reesi, Acinetobacter sp., and members of the Actinomyces and Streptomyces genera, which store up to 80% of dry cell mass as lipids. Other species of bacteria and fungi break lignin down into aromatics.

In some embodiments, the microbes utilized in inoculation are grown in starter cultures using standard procedures. The standard procedures may vary according to the particular species selected.

The resultant lipids may include any molecular forms having a straight-chain hydrocarbon portion. Such lipids are desirable because the straight-chain hydrocarbon portion is relatively easy to convert to vehicle fuel.

Lipids include TAGs and wax esters. Mono- or poly-unsaturated hydrocarbon chains are also found in lipids and are suitable for conversion to alkanes, albeit with the requirement of additional hydrogen to saturate them.

The resultant aromatic compounds include any molecular forms having carbon ring structures. Examples of preferred aromatics include xylenes, methyl benzenes, and others.

In some embodiments, TAG and aromatic production is promoted by maintaining the microbes in a high-carbon, low-nitrogen environment, and providing aeration and/or agitation. As is understood, optimizing the percentage of feedstock carbon converted to TAG or aromatics requires controlling the growth of the microbial culture so as to reduce the carbon consumed by cell replication and metabolic activity and to increase the carbon consumed in producing TAG and aromatics. This can be done by controlling the ratio of non-carbon nutrient to carbon in the feedstock, as well as by controlling other parameters such as pH, temperature, dissolved oxygen, carbon dioxide production, fluid shear, and the like. In some embodiments, one or more measurements of these parameters may be used to determine when to harvest produced TAG. In other words, one or more of these parameters may have a value associated with or which is indicative of desired TAG production.

For example, in some embodiments, fluid shear is controlled by either moving the reactor vessel as a whole (e.g., by rocking it back and forth at a controlled frequency) or by means of mechanical agitators immersed in the fluid (e.g., any of a variety of paddle or stirrer shapes driven by electrical motors at a controlled frequency).

In some embodiments, aeration or oxygenation of the fluid is accomplished by any number of means, including via entrainment of air due to turbulence caused by mechanical agitation of the fluid and via bubbling air, air enriched with oxygen, or pure oxygen through the fluid.

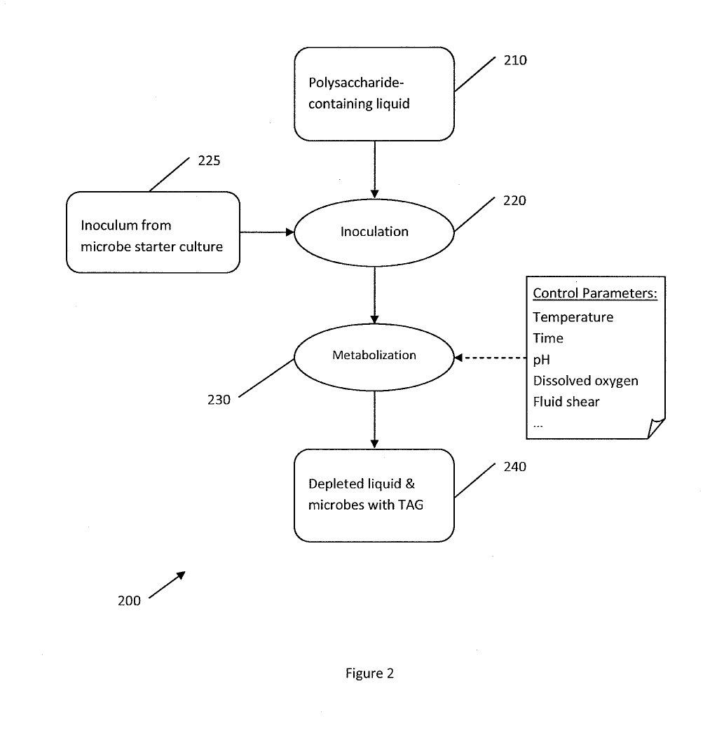

Referring now to FIG. 2, a flow chart of an inoculation and fermentation process 200 in accordance with an embodiment of the invention is shown. The inoculation and fermentation process 200 includes a receiving stage 210 for receiving the liquid output 144 from the pretreatment process 100 and an inoculation step 220 that adds a starter culture 225 of the selected microorganism to the liquid 144 to form a mixture at the inoculation step 220. The selected microorganism may be a single species or strain, or a combination of multiple species or strains.

The inoculation and fermentation process 200 also includes a metabolization step 230, which takes the mixture and controls parameters such as temperature, pH, dissolved oxygen, and fluid shear using appropriate methods known in the art. During this metabolization step 230, the microorganisms proliferate and then metabolize the feedstock, creating intracellular inclusions of lipids. At the end of this stage (e.g., as determined by defined values of one or more of the parameters of time, pH, dissolved oxygen or others) the metabolization is stopped, yielding a depleted fluid 240 with suspended microbes containing lipids.

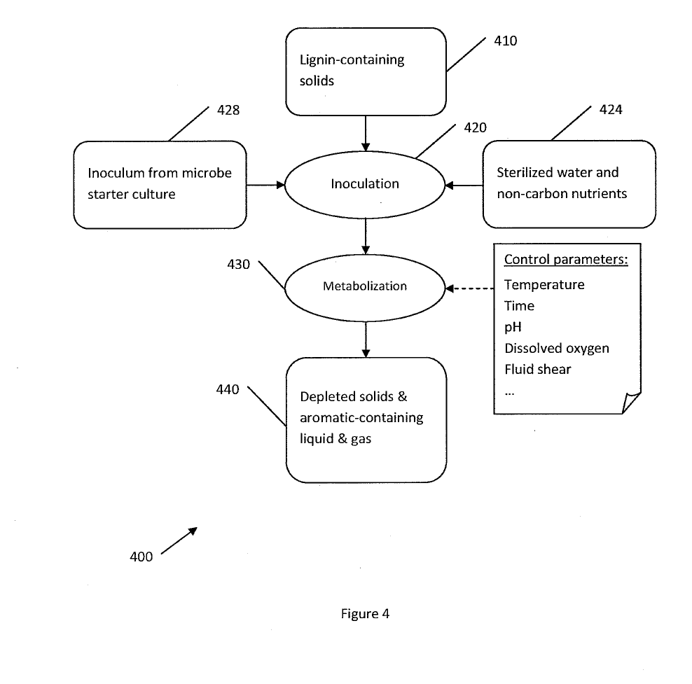

Referring now to FIG. 4, a flow chart of an inoculation and fermentation process 400 in accordance with an embodiment of the invention is shown. The inoculation and fermentation process 400 includes a receiving stage 410 for receiving the solid portion of the pretreated feedstock 148 from the pretreatment process 100 and an inoculation step 420, in which the portion of feedstock 148 is mixed with sterilized water and non-carbon nutrients 424 and a starter culture of specially selected microorganisms suited to decomposing the lignin 428.

The inoculation and fermentation process 400 also includes a metabolization step 430, which takes this mixture and controls parameters such as temperature, pH, dissolved oxygen, and fluid shear using appropriate methods known in the art. During this metabolization step 430, the microorganisms proliferate and then metabolize the feedstock, breaking the lignin down into smaller aromatic compounds that are released into the solution. At the end of this stage (e.g., as determined by defined values of one or more of the parameters of time, pH, dissolved oxygen or others) the metabolization is stopped, yielding a mixture 440 containing depleted solids, microbes, and gas and liquid containing the desired aromatic compounds.

### (3) Harvesting and Product Extraction

The process for extracting product from a digester depends on whether the product is TAG from cellulose breakdown or aromatic hydrocarbons from lignin breakdown. Each is considered in turn. In both cases, however, choosing the proper time to harvest will maximize yield. Measurements such as pH, dissolved oxygen, carbon dioxide production, remaining carbon nutrient concentration, and the like can be used to determine the optimal harvest time.

Harvesting and Extracting TAG

The liquid medium in the digesters provides nourishment to the TAG-producing microbes, allowing the microbes to flourish and reproduce. These microbes store TAG in intracellular structures. The first step, accordingly, is to harvest or collect the cellular biomass from the liquid medium. Some cells tend to form multicellular agglomerations hundreds of micrometers in size, in which case the harvesting may be performed by screening, sieving, centrifugation, or filtration. The result of this step is a mass of cellular matter which typically includes excess water, e.g. wet fermentation product. When the cells tend to remain separate, harvesting may include adding agglomerating agents and other cell separation steps.

In some embodiments, the wet fermentation product is dried after the collecting step. For example, gross excess water may be removed by pressing through a roller press. The product may then be further dried using a vacuum oven, lyophilizer, or other common drying equipment. It should be recognized that when using a vacuum oven, for example, the temperature should be controlled so that TAG is not or is only minimally hydrolyzed. In some embodiments, lyophilizing is selected as the drying means because it has the effect of increasing the surface area to volume ratio of the resulting dry matter, thereby making subsequent extraction quicker. In some embodiments, flash freezing (e.g., via immersion in liquid nitrogen) is used to break up the cell structures, improving efficiency of subsequent extraction.

Because extracted liquids may contain residual nutrients, as well as microbial cells that escaped harvest, this fluid may be recycled. For example, in one embodiment, the fluid (e.g., filtrate) from one production cycle is used as a portion of the starting broth (e.g., liquid medium) of the next production cycle. Because the fluid may also contain metabolites released by the reproducing and digesting microbes, and high metabolite concentration may inhibit the succeeding production cycle, in one embodiment, the recycled fluid is treated to neutralize the metabolites. The recycled fluid may also, in some instances, be sterilized.

Following collection, the cellular matter is exposed to a cell disruptor, e.g., means for extracting the lipid material from within the cells. In some embodiments, the cell disruptor frees lipids from microbe cells using, for example, heat, ultrasound or chemical disruption (lysis) of the cells. In one embodiment, chemical lysis includes utilizing a chloroform-methanol solution to lyse the cells and their internal structures. Without wishing to be bound by any particular theory, it is believed that the methanol disrupts the cell, and the chloroform extracts the lipids. Other chemical solvents, including but not limited to methylene chloride and chloroform-methanol, may also be used in chemical lysis and lipid extraction.

Once the lipids have been released from the intracellular structure, they are separated from the cellular debris. In some embodiments, a mechanical lipid separator is used. For example, a doctor-blade to guide a floating lipid-rich mass from the top of the mixture, a sump to draw heavier components from the bottom of the lipid separator, or other port means depending on the properties of the lipids may be used. Furthermore, in some embodiments, a chemical solvation process may be utilized to provide a higher level of purity of TAG. For example, using light alkane solvents like hexane or heptanes yields a purer TAG than mechanical means because phospholipids and proteins are insoluble in alkanes. Consequently, the resulting TAG may be low in contamination by phosphorus and metals, which is desirable in some fuels.

After extraction of TAG, TAG is converted into hydrocarbons that may then be fractionated to form constituents of gasoline, diesel or jet fuel. Such conversion process is known to those skilled in the art.

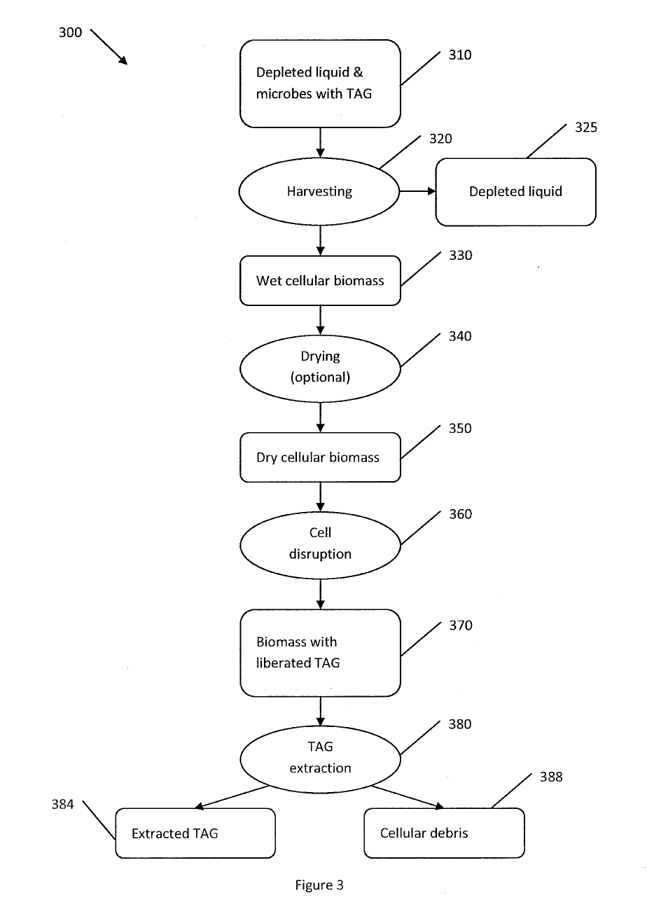

Referring now to FIG. 3, a flow chart of a microbial matter or intermediary product, collection process 300 in accordance with an embodiment of the invention is shown. The microbial collection process 300 includes a receiving stage 310 for receiving the depleted fluid 240 with suspended microbes containing TAG from the inoculation and fermentation process 200 and uses one or more separation technique as described herein to harvest or collect 320 microbial matter or intermediary product 330. In some embodiments, mechanical means such as one or more of filtration, sieving, screening, centrifugation or precipitation, is used to separate the microbial matter 330 from the depleted liquid 325.

In some embodiments, the depleted liquid 325 is recycled as part of the water 134 added to the feedstock in the pretreatment stage 100 of FIG. 1. The depleted liquid 325 may require buffering, not shown, to mitigate the otherwise inhibitory effect of metabolites secreted by the microbes in the metabolization stage 230 of FIG. 2.

The microbial matter or intermediary product 330 consists of wet microbial fermentation product. Accordingly, a drying step 340 may optionally be performed, to speed the extraction process. The drying step 340 may utilize heating in an oven, heating and/or evacuation in a vacuum oven, lyophilization, with or without use of a cryogenic liquid, or other desiccation means. The result of this step 340 is a dry microbial matter or intermediary product 350.

Either the wet matter 330 or the dry matter 350 is then subjected to a cell disruption step 360 that breaks up the cell structures to render the TAG accessible to chemical solvents. The cell disruption step 360 may utilize methods including one or more of mechanical, thermal, or chemical methods. For example, mechanical disruption methods may include one or more of ultrasonic, cutting, pressing, rolling or abrading means. Thermal methods may use heated air or microwave energy, among other means. Chemical means use one of several chemical agents, including but not limited to chloroform, chloroform and methanol, or methylene chloride. The output of the cell disruption step 360 is a biomass with liberated TAG 370. Disrupting chemicals used in this step 360 may be captured, recovered and reused in a closed-cycle system. The microbial collection process 300 also includes a TAG extraction or initial purification step 380. In some embodiments, TAG extraction is performed via chemical solvation, using solvents including short-chain alkanes such as hexane and heptanes. Solvation is followed by decantation, repeated as needed to achieve the required purity of TAG and freedom from contaminants. The output of the TAG extraction step 380 is extracted and purified TAG 384, along with cellular debris 388. Solvents used in this step 380 may be captured, recovered and reused in a closed-cycle system.

As stated above, the dry microbial matter or intermediary product contains the TAG within the microbial cells. The next step simultaneously disrupts the cells and extracts the TAG. It relies on a mixture of solvents:
a. an alcohol-based solvent (such as methanol, ethanol, isopropanol, or the like) to disrupt the cell structures, and
b. a polar organic solvent (such as chloroform, methylene chloride, acetone, or the like) to extract the TAG.

In one embodiment, the solvent comprises a mixture of 10% methanol and 90% chloroform, by volume. The percentages need not be precise.

If the dry microbial matter is dense and leathery, it may be pre-soaked in the solvent mixture for several hours prior to the next step. If it is porous and fluffy, pre-soaking is not needed.

Cell disruption and TAG extraction proceeds by percolating hot solvent mixtures repeatedly through an amount of dry microbial matter. In the laboratory, this can be accomplished by a Soxhlet apparatus. At an industrial scale, the Soxhlet apparatus may be replaced by a system that is more robust and more energy-efficient at large scale. The underlying chemical principle remains the same: repeated exposure of the dry fermentation product to the hot solvents until nearly all the cells are disrupted and nearly all the TAG has gone into solution. In the Soxhlet apparatus, heat is applied to a reservoir of solvent, causing it to boil. The vapor rises until it condenses in a condenser cooled just below the boiling point. The condensate drips into a vessel containing the dry biomass. The hot (and hence chemically more active) solvent level rises to submerge the biomass. A siphon at the top of the vessel completely drains the vessel back into the solvent reservoir every time the liquid in the vessel reaches the top of the bend in the siphon. This process can take several tens of minutes. During this time, the solvent mixture is both breaking down the cell structures and dissolving the TAG (and other intracellular molecules). When the vessel empties into the solvent reservoir, it now clinics the dissolved TAG with it. The cycle of evaporation-condensation-filling-dissolving-siphoning may be repeated until no further significant quantity of TAG is extracted from the biomass. The material collected in the solvent reservoir contains the TAG, now extracted from the microbial cells.

In some embodiments, the reservoir contains TAG, other biomolecules soluble in the polar solvent, and the solvent itself. An evaporation and distillation stage evaporates the solvent out of the mixture and condenses it, recapturing the solvent for reuse. What now remains in the reservoir is called crude TAG, since it may contain impurities.

A refining step includes treating the crude TAG in a solvent made of short-chain hydrocarbons such as heptane or mixtures of heptane with hexane or petroleum ether. One embodiment uses a 1:1 mixture of heptane and low-boiling-point petroleum ether (with boiling point between 40° C. and 60° C.).

In some embodiments, the cellular debris 388 is sent to a gasifier and consumed to produce on-site electricity and/or process heat. The cellular debris 388 may also be used as part of the carbon and non-carbon nutrients in the metabolization stage 230 of FIG. 2. Alternatively, the cellular debris 388 may be collected, processed and sold as other products, such as livestock feed.

As is easily appreciated, TAG produced in accordance with embodiments of the present invention may be used as a liquid fuel suitable for transportation uses. In some embodiments, the fuel product includes saturated non-aromatic hydrocarbon molecules (e.g., straight and branched alkanes) with molecular weights in a predetermined range (e.g., as required by vehicle engines).

In some embodiments, TAG constituents may be used as a substitute for gasoline. In such embodiments, TAG includes constituents in the approximately 6 to 12 carbon range.

In some embodiments, TAG constituents may be used as a substitute for aviation fuel. In such embodiments, TAG constituents include primarily alkanes.

In some embodiments, TAG constituents may be used as a substitute for diesel fuel. In such embodiments, TAG includes alkanes in the 16 to 18 carbon range, and optionally additional minor constituents in the approximately 14 to 20 carbon range.

Table 1 shows exemplary TAG constituents produced by a selected strain of microbes.

TABLE 1
Produced TAG

| | | | | | Glucose/ |
|---|---|---|---|---|---|
| | Chain | Double | Glycerol | | Glycerol |
| Name | length | bonds | Sample A9 | Sample A12 | Sample C2 |
| Palmitic | 16 | 0 | 22.57% | 21.57% | 19.30% |
| Stearic | 18 | 0 | 13.55% | 16.72% | 13.60% |
| Oleic | 18 | 1 | 27.70% | 28.51% | 45.10% |
| Linoleic | 18 | 2 | 30.32% | 26.90% | 17.60% |

| | | | |
|---|---|---|---|
| TOTAL | 94.14% | 93.70% | 95.60% |

As shown in Table 1, the microbes were provided either glycerol or a combination of glucose and glycerol as their carbon source. The main components of this particular TAG product include linoleic acid, oleic acid, stearic acid and palmitic acid. The carbon chain length distribution in Table 1 indicates that any liquid transportation fuel can be refined from the product, with reasonable efficiency. In addition to the major components identified in the Table 1, the TAG includes 1-2% lignoceric acid (24-carbon chains, 0 double bonds), and less than 1% each of fatty acids with carbon chain length X and number of double bonds Y, indicated as (X:Y), as follows: (14:0), (15:0), (16:1), (17:0), (18:3), (20:1), (20:2), (20:4), (22:0).

As is easily appreciated, the product composition may be adjusted, by varying process conditions, to partially offset feedstock variations and to meet application specifications. Depending on product specifications, in some embodiments, the liquid fuel product may contain a proportion of saturated aromatic carbon compounds. For example, jet fuel specifications call for aromatic components comprising between 8% and 25%, by weight, of the total fuel composition.

Extracting Aromatic Compounds

As stated above, extracting product from a digester is different, depending on whether the product is TAG from cellulose breakdown or aromatic hydrocarbons from lignin breakdown. The digester that receives the solid, lignin-rich portion of pretreated feedstock includes water, nutrients and an appropriate inoculum added to break the lignin down into a variety of aromatic compounds. At the end of the fermentation or digestion cycle, the solid mass is a combination of microbes and undigested solid feedstock.

The aromatic compounds are included as part of the liquid and gas phase of the digester output (rather than being stored intracellularly as in TAG production). This is because the microbes break lignin down not primarily to digest it for nutrient value, but to gain access to proteins inside the lignin structures. Thus, the microbes do not absorb and metabolize the lignin breakdown products.

In some embodiments, the solid portion of the digester contents is largely waste that can be disposed of or gasified to produce electricity and process heat. Standard chemical separation and purification processes may be implemented to capture the aromatics from the liquid and gas-phase outputs of the fermentation.

After extraction of the aromatic compounds, the aromatics may then be fractionated by molecular weight. The fractionated aromatics may then be blended with alkanes to form constituents of gasoline, diesel or jet fuel. Such blending process is known to those skilled in the art.

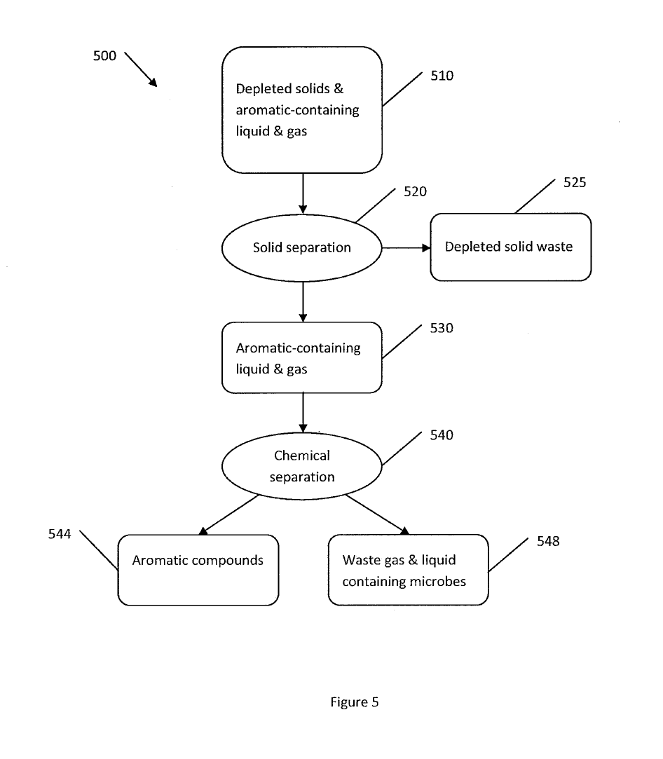

Referring now to FIG. 5, a flow chart of a separation process 500 in accordance with an embodiment of the invention is shown. The separation process 500 includes a receiving stage 510 for receiving the mixture 440 containing depleted solids, microbes, and gas and liquid containing the desired aromatic compounds yielded by the metabolization step 430 of FIG. 4.

The separation process 500 subjects the mixture 440 to a mechanical solids separation step 520. This separation step 520 uses one or more of standard mechanical means such as screening, sieving, centrifugation or filtration to achieve the separation. The separated depleted solids 525 can be sent to a gasifier and consumed to produce on-site electricity and/or process heat. Alternatively, the depleted solids may be collected, processed and sold as other products, such as livestock feed.

The separation step 520 also outputs liquid and gas 530 containing the target aromatic compounds. A chemical separation step 540, using standard chemical processes known in the art, separates aromatic compounds from the others and fractionates them by molecular weight, yielding the aromatic compounds of interest 544. The byproduct of this chemical separation step 540 is the waste gas and liquid 548, which may contain microbial cell bodies. In some embodiments, this waste liquid 548 is recycled to form part of the input water mixture 134 of the feedstock pretreatment stage 130 of FIG. 1.

The production of TAG and aromatic compounds may be associated with or implemented by a cellulose processing plant and/or a bio-refinery producing transportation fuel. The association may be integral, parallel, or separate.

In some embodiments, a cellulose processing plant receives agricultural waste (or other cellulosic material), converts it into TAGs by microbial action, and then extracts intermediates from TAGs that may be converted to fuel. In contrast, a bio-refinery typically receives TAG and aromatic compounds, processes them and blends them into transportation fuels.

In one embodiment, the production of TAG and aromatic compounds is implemented by a cellulose processing plant in parallel with a bio-refinery. In such an embodiment, glycerol produced by the bio-refinery is used to generate further lipids, and then either convert the lipids into fuel or pass the lipids to the bio-refinery plant which converts the lipids to fuel.

In another embodiment, the production of TAG and aromatic compounds is implemented by a cellulose processing plant integrated with a bio-refinery. In such an embodiment, the cellulose processing system is utilized to produce glycerol. For example, the same vessel may contain both the cellulose digestion mixture and the glycerol consumption mixture intermingled. The microbes for cellulose digestion and glycerol consumption may be intermingled if they are compatible. It is envisioned that the same microbe may perform both cellulose digestion and glycerol production simultaneously. Similarly, a single combined lipid product may be recovered from both processes.

In another embodiment, the production of TAG and aromatic compounds is implemented by a cellulose processing plant separate from a bio-refinery. In such an embodiment, the glycerol processing is separate from the cellulose processing. In one example, the glycerol feed may be reduced all the way to the fuel product. Alternatively, the glycerol feed may provide lipids as an intermediate product, with fuel production being completed at the separate bio-refinery or chemical refinery. In some embodiments, alkanes are extracted from TAGs and recycled in the glycerol processor to generate further fuel. This process may be repeated in cyclical fashion until the feed material is exhausted.

From the above description, a method in accordance with embodiments of the present invention, include a series of steps. These steps include one or more of the following:
(1) Receiving and pretreating cellulosic feedstock;
(2) Optionally, adding glycerol obtained as a co-product of TAG transesterification;
(3) Separating the pretreated feedstock into liquid and solid phases;
(4) Inoculating the liquid phase with microbes that are capable of converting the carbon into lipids, then allowing the microbes to do so;
(5) Harvesting the resulting microbial matter from the liquid;
(6) Extracting the lipids for subsequent conversion into fuels.
(7) Mixing the solid-phase pretreated feedstock with water and nutrients, then inoculating it with microbes capable of attacking the lignin and converting it into aromatic compounds;
(8) Separating the resulting aromatics from the liquid and gas phases of the digester output;
(9) Recycling the remaining solid-phase matter as a co-product or as feed for gasification and conversion to heat and electricity;
(10) Recycling the liquid-phase matter as broth for the next batch of feedstock and fermentation.

Additionally, in some embodiments, the pretreatment process 100, as shown in FIG. 1, leaves considerable cellulose and hemicellulose in the solid phase or portion 148. In such embodiments, the solid-phase feedstock 148 is inoculated with a consortium of microbes that includes species to digest the cellulose and hemicellulose and produce intracellular TAG as well as species to break down the lignin and secrete aromatic molecules in step 420. Following the metabolization step 430, the aromatic compound separation 520 proceeds as indicated in FIG. 5, but the solid phase extract 525 is no longer mere waste or recycling material, but is subjected to the TAG extraction process 330 of FIG. 3.

In some embodiments, the liquid-solid separation step 140 at the end of the feedstock pretreatment process 100 of FIG. 1 is absent. In such embodiments, the unseparated feedstock is inoculated with a consortium of microbes capable of digesting both liquid and solid phases, the aromatic compounds are separated as shown in FIG. 5, and the TAG is extracted as shown in FIG. 3.

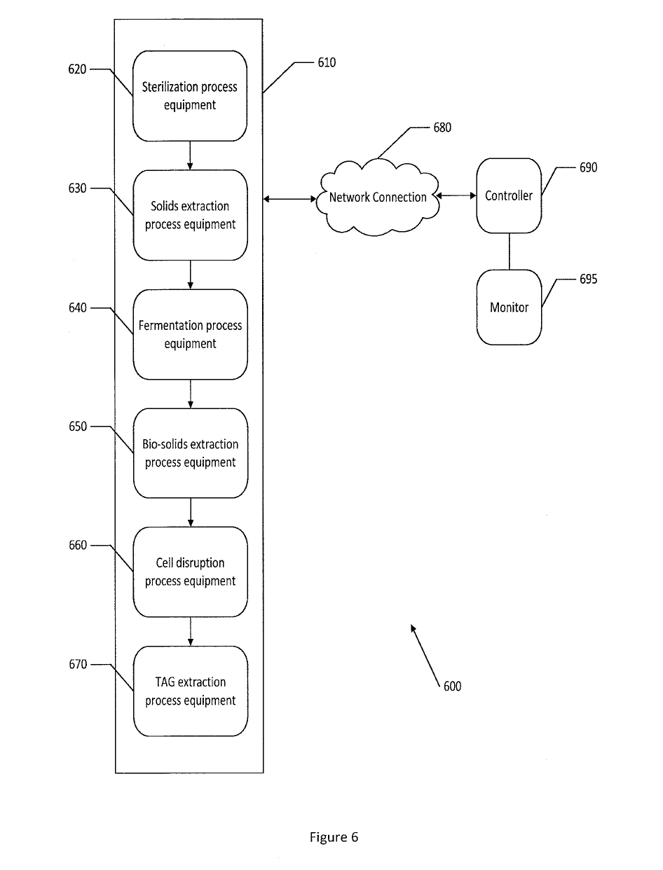

Turning now to FIG. 6, a system 600 for producing TAG in accordance with an embodiment is shown. System 600 includes a processing plant or facility 610 in communication with a controller 690. In one embodiment, processing plant 610 communicates with controller 690 via a network connection 680. Network connection 680 may be wireless or hard-wired.

In some embodiments, controller 690 provides operating instructions for processing plant 610's operating conditions. Controller 690 may receive information from processing plant 610 and utilize the information as feedback to adjust operating instructions to processing plant 610.

In one embodiment, the operating conditions may be presented on a monitor or display 695 and a user may interact with the operating conditions via a user interface. The monitor 695 may be in the form of a cathode ray tube, a flat panel screen or any other display module. The user interface may include a keyboard, mouse, joystick, write pen or other device such as a microphone, video camera or other user input device.

Processing facility 610 includes sterilization process equipment or sterilizer 620, solids extraction process equipment or solids extractor 630, fermentation process equipment or fermentor 640, bio-solids extraction process equipment or bio-solids extractor 650, cell disruption process equipment or cell disruptor 660 and TAG extraction process equipment or TAG extractor 670. In some embodiments, controller 690 is in communication with fermenter 640 and provides/controls the operating conditions of fermentor 640.

Sterilization process equipment 620 and solids extraction process equipment 630 together perform the cellulosic feedstock pretreatment process 100 of FIG. 1. Fermentation process equipment 640 performs the inoculation and fermentation process 200 of FIG. 2. Bio-solids extraction process equipment 650, cell disruption process equipment 660 and TAG extraction process equipment 670 together perform the microbial biomass collection process 300 of FIG. 3.

Those of skill will appreciate that the various illustrative logical blocks, modules, and algorithm steps described in connection with the embodiments disclosed herein can often be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the design constraints imposed on the overall system. Skilled persons can implement the described functionality in varying ways for each particular application, but such implementation decisions should not be interpreted as causing a departure from the scope of the invention. In addition, the grouping of functions within a module, block or step is for ease of description. Specific functions or steps can be moved from one module or block without departing from the invention.

The various illustrative logical blocks and modules described in connection with the embodiments disclosed herein can be implemented or performed with a general purpose processor, a digital signal processor (DSP), application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

The steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium. An exemplary storage medium can be coupled to the processor such that the processor can read information from, and write information to, the storage medium. In the alternative, the storage medium can be integral to the processor. The processor and the storage medium can reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. For example, while the feedstock received by a cellulose processing plant has been referred to as containing cellulosic material, any type of feedstock which may yield alkanes and/or aromatic compounds may be used. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1-30. (canceled)

31. A method for producing lipids comprising:
(a) receiving a feedstock including biological matter;
(b) inoculating the feedstock with microbes selected from the group consisting of bacterial species, fungal species or a combination thereof, wherein the microbes convert cellulose, hemicellulose or glycerol into lipids upon fermentation with the feedstock;
(c) fermenting the inoculated feedstock, thereby producing lipids.

32. The method of claim 31, wherein the microbes comprise Trichoderma reesi, Acinetobacter sp., or members of the Actinomyces and Streptomyces genera.

33. The method of claim 31, wherein the feedstock comprises cellulose, hemicellulose or a combination thereof.

34. The method of claim 33, wherein the feedstock comprises sawdust, wood chips, algae, or municipal solid waste.

35. The method of claim 31, wherein the feedstock is fortified with glycerol.

36. The method of claim 31, further comprising pretreating the feedstock with at least one of mechanical pretreatment, thermal-chemical pretreatment, sterilization, ultraviolet irradiation, pasteurization, filtration and separation prior to inoculating the feedstock.

37. The method of claim 36, wherein the pretreatment makes the feedstock more amenable to microbial digestion.

38. The method of claim 36, wherein the wherein the pretreatment increases the ratio of surface area to volume of the feedstock.

39. The method of claim 36, wherein the mechanical pretreatment comprises chopping, shredding, grinding or a combination thereof.

40. The method of claim 36, wherein pretreating comprises separating the feedstock into a liquid phase and a solid phase.

41. The method of claim 40, wherein the liquid phase and the solid phase are fermented separately in separate vessels.

42. The method of claim 31, wherein fermenting comprises agitation, aeration or oxygenation.

43. The method of claim 31, wherein the lipids comprise triacylglycerides.

44. The method of claim 43, wherein the triacylglycerides comprise linoleic acid, oleic acid, stearic acid, palmitic acid or a combination thereof.

45. The method of claim 31, wherein fermentation comprises reproduction of the microbes and the method further comprises harvesting a cellular biomass comprising the reproduced microbes.

46. The method of claim 44, wherein fermentation comprises reproduction of the microbes and the method further comprises harvesting a cellular biomass comprising the reproduced microbes and recovering the lipids produced by the microbes from the cellular biomass.

47. The cellular biomass produced according the method of claim 45.

48. The lipids produced according the method of claim 14.

49. The lipids of claim 48, wherein the lipids comprise triacylglycerides.

50. The lipids of claim 49, wherein the triacylglycerides comprise linoleic acid, oleic acid, stearic acid, palmitic acid or a combination thereof.
